(19) United States
(12) Patent Application Publication
(10) Pub. No.: US 2019/0069770 A1
(43) Pub. Date: Mar. 7, 2019

(54) ILLUMINATED DENTAL MIRROR

(71) Applicant: Raydent Corporation, Greenville, MI (US)

(72) Inventors: Paul L. Bourget, Kentwood, MI (US); Harold W. Brunt, Jr., Grand Rapids, MI (US); Donald P. McConnell, Grand Rapids, MI (US); Robert R. Phillips, Comstock Park, MI (US); Justin E. Sobecki, Rockford, MI (US); David J. Ippel, Jenison, MI (US); Brian S. Zatzke, Spring Lake, MI (US)

(21) Appl. No.: 15/695,331

(22) Filed: Sep. 5, 2017

(51) Int. Cl. A61B 1/247; A61B 1/07; A61B 1/00; A61B 1/06

(52) U.S. Cl. CPC A61B 1/247; A61B 1/07; A61B 1/0669; A61B 1/00105; A61B 1/00103

(57) ABSTRACT

An illuminated dental mirror includes an elongated body that has a handle portion and a tip portion. A light source is disposed within the elongated body and is operable to emit light longitudinally along the elongated body to a deflector disposed at an interior chamber of the tip portion. The deflector is configured to redirect light laterally out of the tip portion of the elongated body. A disposable mirror head is removably attached over the tip portion. The mirror head includes a base portion and a mirror portion. The base portion of the mirror head has a translucent material configured to transmit the light redirected by the deflector to a desired area outside the mirror head. The mirror portion that extends at an angle from the base portion and comprises a mirror that is configured for a user to view the desired area illuminated by the light source.

### FIELD OF THE INVENTION

The invention relates to dentistry and more specifically to a hand held dental mirror, commonly known for dental inspection.

### BACKGROUND OF THE INVENTION

One may consider the mouth to be cave-like and typically poorly illuminated. Various lighting equipment is known to focus or project light from a source toward a selected object. This lighting equipment may be mounted on a stand, an arm, or another mechanism having a similar function for positioning by a user. Lighting equipment may alternatively be mounted on a user's head such that illumination may be maneuvered by the user manipulating his head. These lighting sources may easily be blocked by the user's hands and the like, however.

### SUMMARY OF THE INVENTION

The present invention provides an illuminated dental mirror that may comprise a handle with a light source in a body of the handle. A light rod or lens may direct light from the light source to a terminal end of a tip of the handle body. The deflector is configured to direct light from the light rod and in a predetermined direction. A mirror head may be provided and adapted to slide over the tip of the handle body in sheathing engagement. The mirror head includes a dental inspection mirror and a translucent portion that allows light to be directed outward from the tip toward a field of illumination in agreement with a field of view of the mirror. After use of the mirror head, it may be removed for cleaning or disposal and replaced with another mirror head that has a sanitary outer surface for use with another patient.

According to one aspect of the present invention, a dental mirror includes an elongated body that has a handle portion and a tip portion. A light source is disposed within the elongated body and is operable to emit light longitudinally along the elongated body to a deflector disposed at an interior chamber of the tip portion. The deflector is configured to redirect light laterally out of the tip portion of the elongated body. A disposable mirror head is removably attached over the tip portion. The mirror head includes a base portion and a mirror portion. The base portion of the mirror head has a translucent material configured to transmit the light redirected by the deflector to a desired area outside the mirror head. The mirror portion that extends at an angle from the base portion and comprises a mirror that is configured for a user to view the desired area illuminated by the light source.

According to another aspect of the present invention, a dental mirror includes an elongated body that has a handle portion and a tip portion with a hollow interior chamber. A light source is disposed within the elongated body and is operable to emit light toward a distal end of the tip portion. A lens member is coupled with the light source and is disposed within the interior chamber of the tip portion. The lens member is configured to transmit light longitudinally along the elongated body from the light source toward the distal end of the tip portion. A deflector is disposed at the interior chamber of the tip portion and is configured to redirect light transmitted from the lens member laterally out of the tip portion. A mirror head is removably attached over the tip portion and includes a base portion and a mirror portion. The base portion has a translucent material that is configured to transmit the light redirected by the deflector. The mirror portion of the mirror head extends at an angle from the base portion and includes a mirror that is configured for a user to view an area illuminated by the light redirected by the deflector through the base portion. The mirror head may also include an attachment feature that removably engages the elongated body in an engaged position that secures and prevents rotation of the mirror head relative to the elongated body.

According to yet another aspect of the present invention, a method of using an illuminated dental mirror includes providing an elongated body that has a handle portion and a tip portion. A light source is disposed within the elongated body and is operable to emit light longitudinally along the elongated body to a deflector disposed at an interior chamber of the tip portion. A disposable mirror head is removably attached over the tip portion. A base portion if the mirror head includes a translucent material that is configured to transmit the light redirected laterally by the deflector to a desired area outside the mirror head. When attached in an engaged position, a mirror portion of the mirror head that extends at an angle from the base portion is positioned for a user to view the desired area illuminated by the light source.

These and other objects, advantages, purposes, and features of the present invention will become apparent upon review of the following specification in conjunction with the drawings.

### BRIEF DESCRIPTION OF THE DRAWINGS

FIG. 1 is an upper perspective view of an illuminated dental mirror, showing a head thereof as transparent in accordance with one embodiment of the present invention;

FIG. 2 is an exploded perspective view of an illuminated dental mirror having an alternative coupling and taken from an opposite end from the dental mirror of FIG. 1;

FIG. 3 is a side elevation view of a light rod lens of the illuminated dental mirror shown in FIG. 2;

FIG. 4 is a cross section view of the light rod lens, taken along an centerline axis thereof;

FIG. 5 is an end elevation view of the head of the illuminated dental mirror of FIG. 2;

FIG. 6 is a front elevation view of the head of the illuminated dental mirror of FIG. 2;

FIG. 7 is a cross section view of the head, taken along line A-A shown in FIG. 6;

FIG. 8 is a cross section view of the head attached on a cap of the illuminated dental mirror of FIG. 2, showing light being emitted from the cap and through the head;

FIG. 9 is a perspective view of a section of the illuminated dental mirror of FIG. 8 with a fragmented portion of the head removed to show a the coupling between the head and the cap;

FIG. 10 is a perspective view of the head with the fragmented portion removed as shown in FIG. 9;

FIG. 11 is a perspective view of another portion of the coupling between the head and the cap of FIG. 2, shown in a slightly separated condition;

FIG. 12 is a perspective view of an additional embodiment a head with the fragmented portion removed, showing an alternative coupling;

FIG. 13 is a perspective view of yet an additional embodiment a head with the fragmented portion removed, showing another alternative coupling;

FIG. 14 is an upper perspective view of the illuminated dental mirror of FIG. 1, showing the head uncoupled and showing another alternative coupling in a slightly separated condition;

FIG. 15 is a side elevation view of a section of the illuminated dental mirror of FIG. 14;

FIG. 16 is a perspective view of the head of the illuminated dental mirror of FIG. 14 with a fragmented portion removed to show a dog on an interior of the head;

FIG. 17 is a cross section view of the cap of the illuminated dental mirror taken along line B-B of FIG. 15;

FIG. 18 is the cross section view of FIG. 17 with the cap seated in the head in sheathing engagement and released from a coupled condition;

FIG. 19 is the cross section view of FIG. 18 with the head rotated relative to the cap, to a coupled condition;

FIG. 20 is a perspective view of an additional embodiment of an illuminated dental mirror, showing a head thereof attached to a cap of a base;

FIG. 21 is another perspective view of the illuminated dental mirror of FIG. 20;

FIG. 22 is a front elevation view of the illuminated dental mirror of FIG. 20;

FIG. 23 is a perspective view of the illuminated dental mirror of FIG. 20, showing the head thereof detached and exploded away from a cap of a base;

FIG. 24 is another perspective view of the illuminated dental mirror of FIG. 23;

FIG. 25 is a front elevation view of the illuminated dental mirror of FIG. 23; and

FIG. 25A is a perspective view of a section of the illuminated dental mirror of FIG. 25. showing the coupling portions of the head exploded away from the cap of the base.

### DETAILED DESCRIPTION OF THE PREFERRED EMBODIMENTS

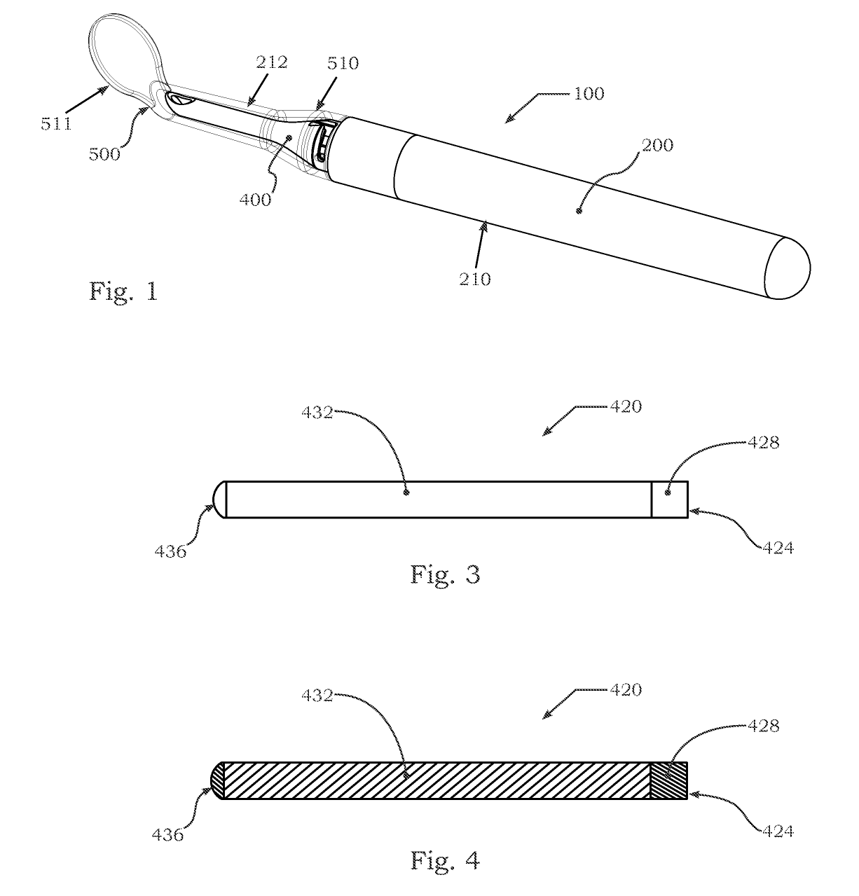

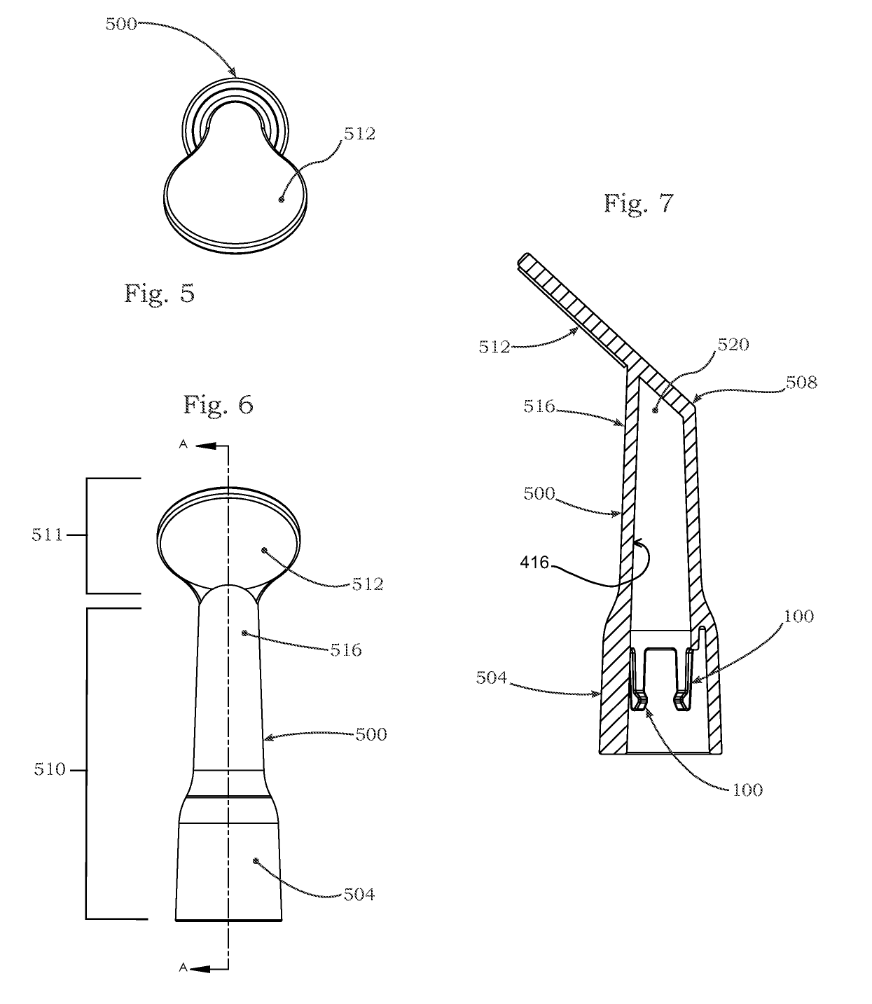

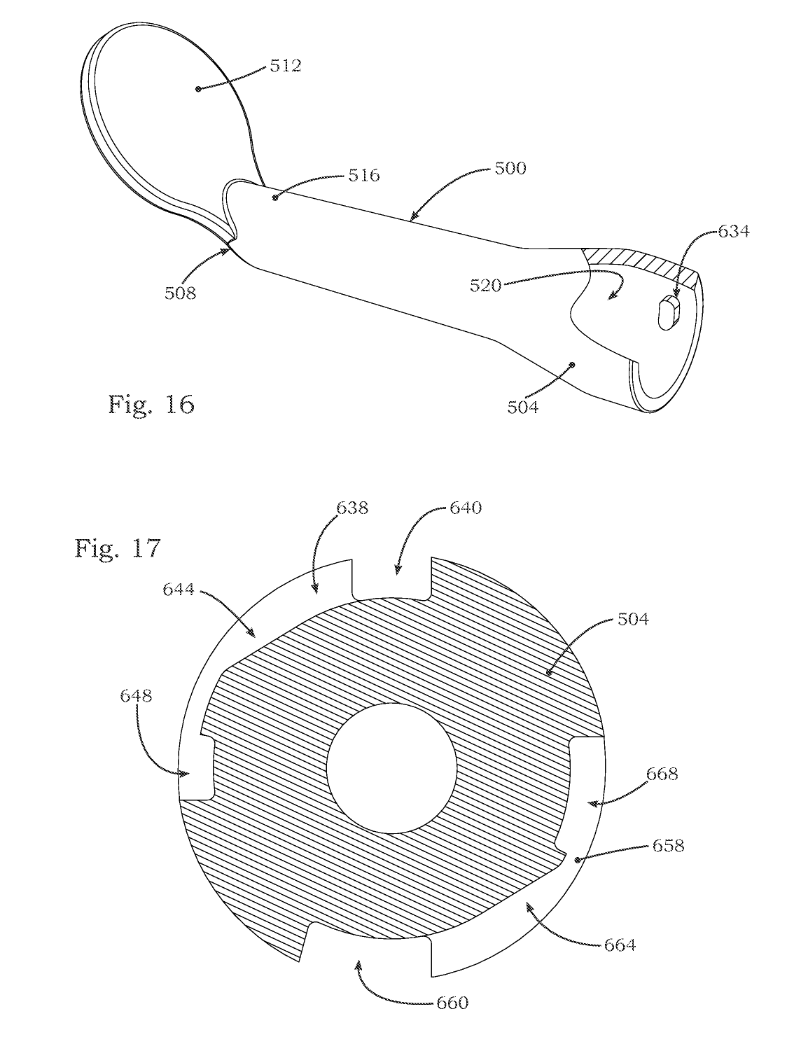

Referring now to the drawings and the illustrative embodiments depicted therein, an illuminated dental mirror 100 includes an elongated body 200 that has a handle portion 210 and a tip portion 212, such as shown as a cap 400 in FIGS. 1-19. A light source 300 is disposed within the elongated body 200 and is operable to emit light longitudinally along the elongated body 200 to a deflector 444, such as a mirror or polished surface or the like, disposed at an interior chamber of the tip portion 212. The deflector 444 is configured to redirect light laterally out of the tip portion 212 of the elongated body 200. A disposable mirror head 500 is removably attached over the tip portion 212. The mirror head 500 includes a base portion 510 and a mirror portion 511. The base portion of the mirror head 500 has a translucent material configured to transmit the light redirected by the deflector 444 to a desired area outside the mirror head 500. The mirror portion 511 extends at an angle from the base portion 510 and comprises a mirror 512 that is configured for a user to view the desired area illuminated by the light source 300. Optionally, the mirror head 500 may include an attachment feature that removably engages the elongated body 200 in an engaged position that secures and prevents rotation of the mirror head 500 relative to the elongated body 200.

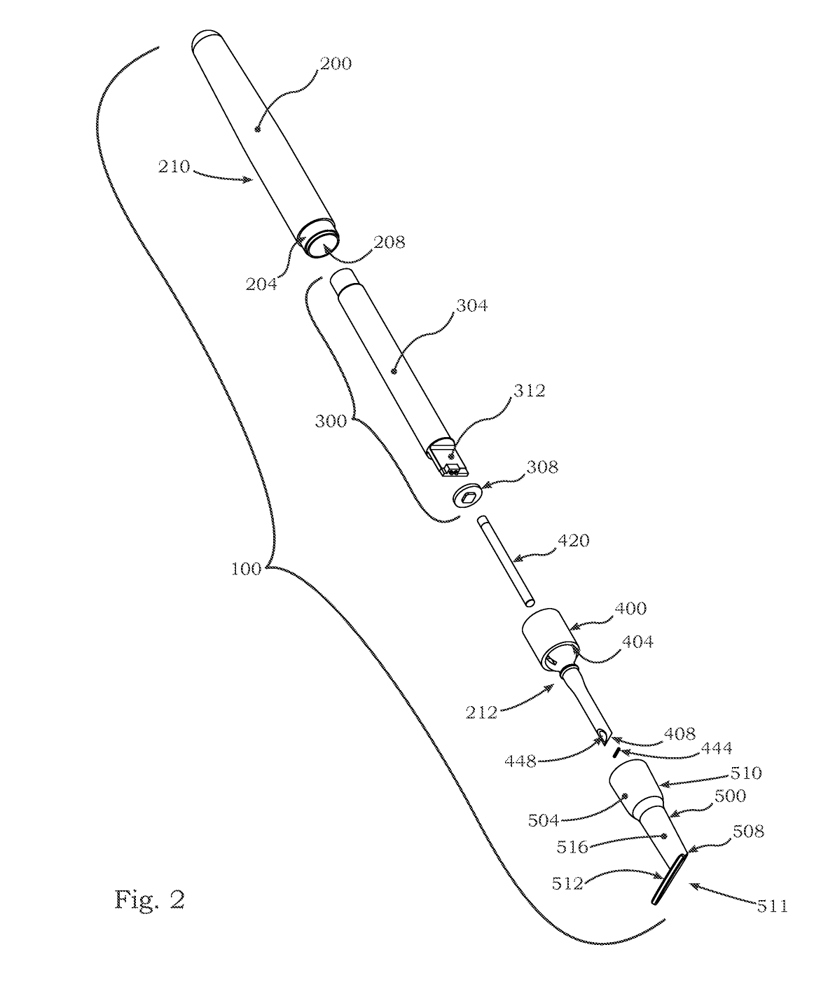

The handle portion 210 of the elongated body 200 may at least partially define a handle or gripping surface, by which the dental mirror 100 may be held and manipulated by a user as is commonly understood. Further, the body may be constructed of any desirable material, including, wood, metal, and plastic as well as composites thereof and by any fabrication method suitable to the selected material as is or may be known. Preferably, however, at least the outer surface of the body is made of a polymer or metal material capable of being sanitized, such as stainless steel, aluminum, or other metals and alloys. The handle portion 210 of the body shown in the illustrated embodiment is an elongated tubular member with two opposing ends, where one end is considered a top 204 and the opposing bottom end is generally enclosed, such as by the illustrated dome-shaped cap or an integrally formed enclosure or the like. Thus, a cavity 208 within the handle portion 210 may be open through the top, such as shown in FIGS. 1 and 2.

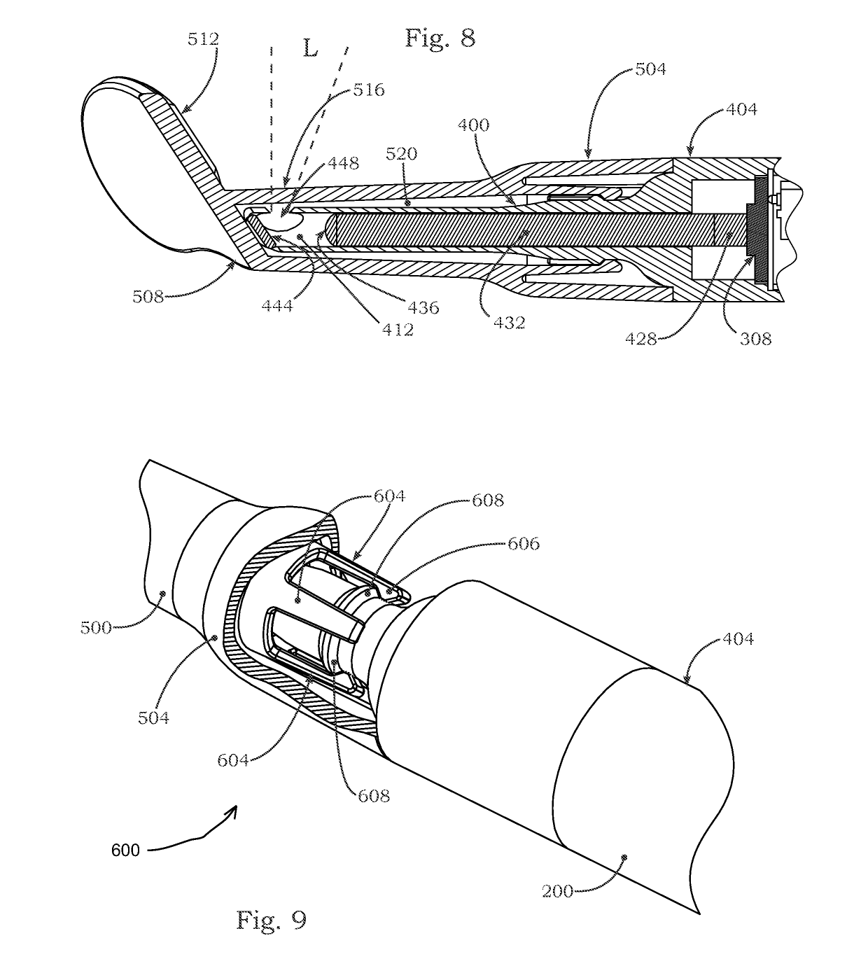

The light source 300 is preferably located at least partially in the cavity 208 and adapted to emit a light “L” out from the top 204 of the handle portion 210 of the body 200, such as longitudinally along and generally in alignment with the longitudinal extent or axis of the handle portion 210 of the body. The light source may be electrically connected to or incorporated in some manner with an energy storage apparatus 304, such as a battery or the like, as shown in FIGS. 2 and 8. The light source 300 may also comprise a light generating device 308, such as a lamp or light emitting diode or diodes or the like, as the light generating device 308 illustrated in FIG. 2 having a light emitting diode disposed on a circuit board. Further, an arrangement of control or operating circuit 312 to actuate or otherwise manipulate the light source may be incorporated in the light source, such as with circuitry disposed at the associated circuit board. The specific makeup and construction of light source or sources may vary from the illustrated arrangement to similarly direct light longitudinally along the handle portion 210.

With respect to dental inspection, it is preferred that the light source emit a white light, although a user's preference may alternatively indicate illumination with a different illumination color, such as light ranging between blue and yellow tones. Thus, it is contemplated that the light source may comprise one or more light sources that have an LED-PCB assembly that is operable to produce such a variable and adjustable spectrum of illumination, and further various other light generating devices may be substituted within the scope of the present invention.

The tip portion 212 of the elongated body 200 is provided with a hollow interior chamber that interconnects with the cavity 208 extending within the handle portion 210 of the elongated body 200. Thus, the light source 300 that is disposed within the elongated body 200 is arranged to emit light through the interior chamber and toward a distal end of the tip portion 212, which is disposed away from the handle portion 210. A deflector may be disposed at the interior chamber of the tip portion 212 and configured to redirect light transmitted from the light source laterally out of the tip portion 212. Specifically, the illustrated tip portion 212 includes a sidewall that has an aperture extending to the hollow interior chamber for transmitting light laterally from the elongated body. As such, the aperture and deflector 444 in the tip portion 212 are arranged to transmit light in a focused area that does not directly contact the mirror portion 511 of the mirror head 500, so as to prevent the user from inadvertently viewing glare from the light source that otherwise may be visible from the aperture when viewing the mirror 512.

With further reference to FIGS. 1, 2, and 6, the tip portion 212 is preferably an elongated member, which is shown as a cap with two opposing ends and a length defined between the two ends. A base 404 of the cap may connect, such as via threaded engagement, with the top 204 of the handle portion to enclose the cavity therein along with the chamber within the tip portion. A distal or terminal end 408 of the tip portion 212 or cap may extend from the base 404, away from the top 204, to the other opposing end of the cap. Similar to the elongated body 200, the tip portion may generally be constructed of any desirable material with any suitable method, although light transmission may be of consideration in selecting a material.

The interior of the cap 400 is preferably adapted to conduct light from the light source 300 to the terminal end 408 of the cap. Thus, the chamber 412 may be defined in the cap to increase efficient transmission of light from the light source to the terminal end 408. This may be accomplished with the chamber being constructed as a lens or the like for efficient light transmission. Alternatively, the chamber may be a void in which a separate lens or light rod 420 may be located, such as shown in the illustrated embodiment.

As shown in FIGS. 2-4, a lens member or light rod 420 may be coupled with the light source and extend within the interior chamber of the tip portion. The lens member is configured to transmit light longitudinally along the elongated body from the light source toward the distal end of the tip portion. The light rod 420 may utilize a light transmitting material in construction of the cap 400 to transmit light from the light source 300 to the end 408. One may also consider the chamber 412 as a void as the light rod 420. Further, polishing a wall 416 of the chamber or the like to minimize light loss through the cap may define the light rod (FIG. 7). More preferably, a rod of a material that provides columniation of the light may be seated in the chamber 412. For example, construction of the light rod or lens 420 may be a compound lens such as doublet (two part) or triplet (three part) lenses, generally shown in the drawing.

In considering the geometry, the optics, of the lens 420 one seeking to implement the invention may also consider an interface between the lamp 308 and a receiving end 424 of the lens. One factor of consideration may be a dispersion pattern of light from the lamp, configuring this pattern, or shaping the receiving end of the lens rod 420 accordingly to capture the light from the lamp as desired. Another interface factor may be whether the lens should contact or be spaced from the lamp. A further factor may be whether an interface facilitating material between the lamp and the lens is desirable. One may consider other aspects of optical interfacing as are known in the art of optics and light transmission.

Again, one configuration of various available configurations regarding the light rod 420, is a three part lens (FIGS. 2-4 and 8). A first portion 428 of the lens at the receiving end 424 may be designed to deflect diverging light from the lamp 308, toward being parallel with the light rod. A second portion 432 of the lens may be designed for clean linear transmission of light, similar to preferences for fiber optic data transmission lines. A third portion 436 of the lens may be designed to focus the light for incidence upon a deflector 444 and emission in a desired or predetermined direction and pattern, discussed further below.

The deflector 444 may be included in the cap 400, at the terminal end 408. The deflector preferably redirects light from the light rod 420 to a pre-selected direction for the ultimate illumination purpose of the invention of clear dental inspection, discussed further below. The deflector may be effected with a mirror device or a mirror or other reflective treatment applied appropriately at the terminal end 408. An emitting lens 448 may also be provided at the terminal end 408 at the aperture, such that light “L” is emitted from the cap in the predetermined direction (FIGS. 2 and 8). The emitting lens may include an optical element that focuses emitted light, such as to focus the light to a desired area within the patient's mouth.

The mirror head 500 is removably attached over the tip portion 212, where the mirror head 500 may include a mirror portion 511 that extends at an acute angle from a base portion 510 of the mirror head 500. The mirror portion 511 of the mirror head 500 comprises a mirror 512 that is configured for a user to view the area illuminated by the light redirected by the deflector through the base portion. The mirror portion 511 may optionally comprise a convex curved surface that is configured to provide a magnified viewing area for the user. The base portion 510 of the mirror head 500 has at least a section that includes translucent or transparent material that is configured to transmit the light redirected by the deflector outward to the desired area of the illumination. The base portion of the mirror head includes a recess that corresponds to at least a portion of the tip portion and extends toward the mirror portion for covering the tip portion of the elongated body. Optionally, the tip portion may include a diameter that is smaller than a diameter of the handle portion, such that the mirror head may engage the elongated body in a manner where the outer surface of the mirror head is substantially aligned with the outer surface of the handle portion of the elongated body.

The mirror head 500 is also preferably an elongated member with two opposing ends. As noted regarding the elongated body, the head 500 may generally be constructed of any desirable material with any suitable method, although light transmission for at least a portion of the mirror head may be of a consideration in selecting a material so not to substantially obstruct the light emitting from the light source. Again, a head base 504 may be provided at one end of the head and be adapted to couple with the cap 400. A recess 520 extends into the head base 504 from the base 504 and toward a tip portion 508 at the other end of the head. More specifically, the recess preferably corresponds to at least a portion of the elongated body of the cap from the terminal end 408 and toward the cap base 404, such that the cap may slide into the head in sheathing engagement, as a sword may slide into its sheath. Optionally, the base portion of the mirror head may have a longitudinal length that is configured to extend into a patient's mouth and position the mirror portion adjacent to a patient's tooth, where the base portion has an impermeable outer surface that is configured to prevent a patient's saliva from contacting the covered tip portion of the elongated body.

The mirror 512 may extend from the head 500 and more specifically from the tip portion 508 at an acute angle relative to a length of the head from the base 504 to the tip 508. The mirror may also be preferably rotationally oriented, and fixed when engaged relative to the cap 400, such that a field of view through the mirror preferably coincides with the light emitted from the cap. A translucent portion 516 of the head aligns with the cap emitting lens 448 and is adapted to pass light emitted from the cap emitting lens (FIG. 8). The translucent portion 516 may also simply be an aperture or may include an optical element that further focuses emitted light as with the cap emitting lens. While either of the cap emitting lens and the portion 516 may be an aperture, at least one may preferably further focus emitted light.

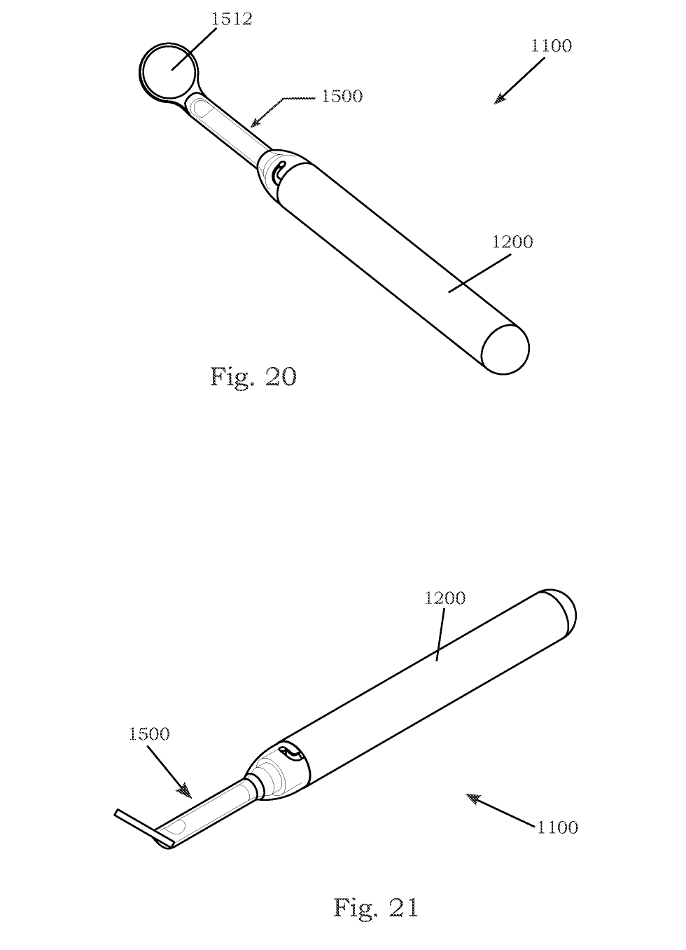

A coupling 600 (FIG. 9) releasably, though securely, couples the head with the cap. The coupling may include an attachment feature at the mirror head that removably engages the elongated body in an engaged position that secures and prevents rotation of the mirror head relative to the elongated body. As shown in FIG. 20, the attachment feature comprises a groove that engages a cooperating dog protruding laterally from the elongated body. The illustrated grove defines a substantially L-shaped groove with a first leg extending longitudinally along the mirror head and a second leg partially circumscribing the base portion of the mirror head.

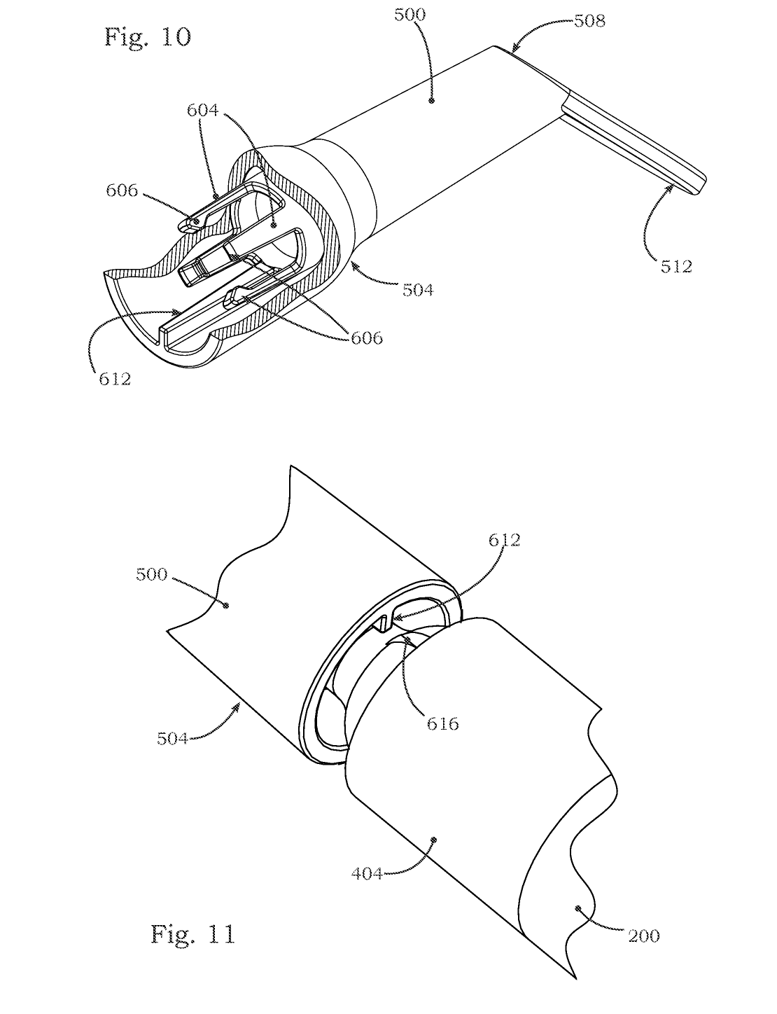

An alternative press-fit coupling may use a latch, such as shown in FIGS. 9 and 10, with at least one and more preferably three resilient fingers 604 for enhanced structural stability that may be provided interior to the head base 504. The at least one finger may define at least one coupling dog 606, respectively. Each dog registers with the catch such that the head securely and releasably couples with the cap (FIGS. 9 and 10). The cooperating catch may be provided as an annular external ring 608 at the cap base 404. A rotational alignment key 612 is preferably defined by one of the head base and the cap base with a mating keyway 616 defined by the other (FIGS. 10 and 11). Without a keying device, the mirror 512 may not necessarily align with light “L” emitted from the cap 400 at the lens 448. Thus, the key 612 and the keyway may preferably be indexed relative to the emitting lens 448 of the cap and with the mirror, respectively.

Provision of the key 612 and the keyway 616 (FIGS. 10 & 11) assure that the cooperating geometry of each of the head interior and of the cap exterior are rotationally asymmetric in the first order, although other embodiments of rotational asymmetry of the first order are anticipated, as is understood by one having ordinary skill in the art. The one having skill in the art further understands that rotational alignment of the head 500 with the cap 400 such that a field of view through the mirror 512 preferably coincides with the light “L” emitted from the cap 400 may be provided by other means, not shown and anticipated in the invention.

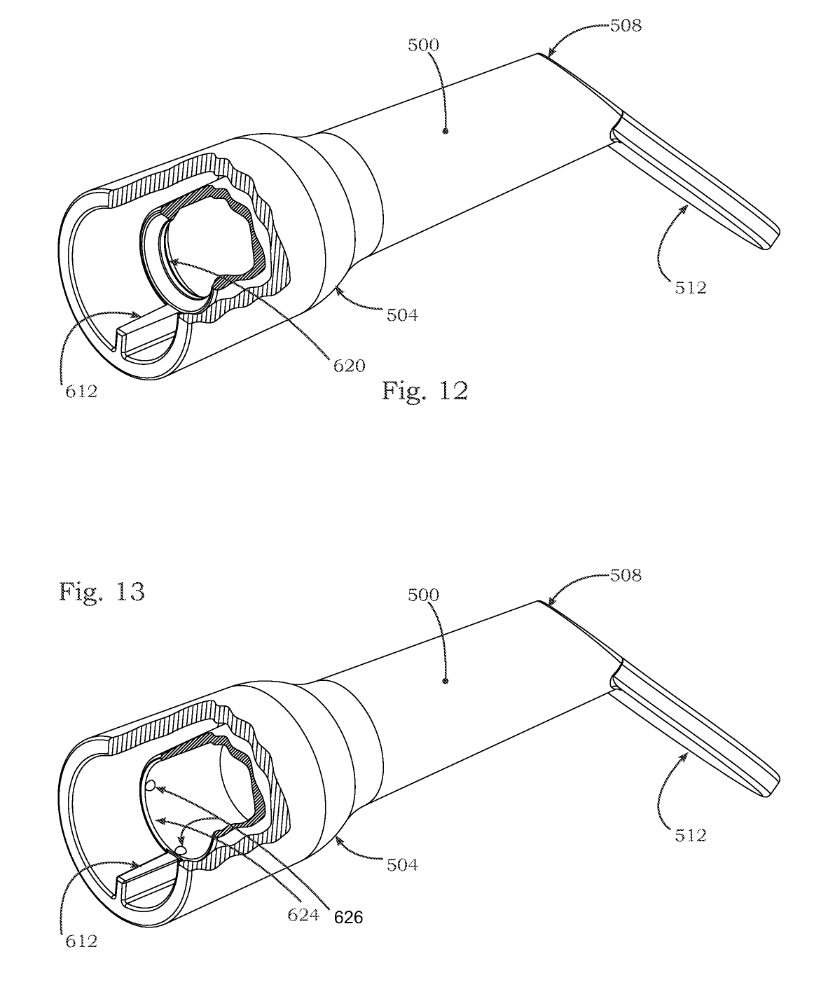

Another alternative press-fit coupling may use an annular ring latch 620 (FIG. 12). The cooperating catch may remain as an annular external ring 608 at the cap base 404 (FIG. 9). One having ordinary skill in the art may also consider that a reversal of elements (not shown) may be employed in which fingers similar to fingers 604, may be provided at the cap base 404. The key 612 and keyway 616 may be utilized with the ring latch 620.

A third, alternative press-fit coupling may use an annular ring latch 624 in which the annular ring dog of ring latch 620 is replaced with at least one discrete dog 626 (FIG. 13). Again, the key 612 and keyway 616 may be utilized with the ring latch 620.

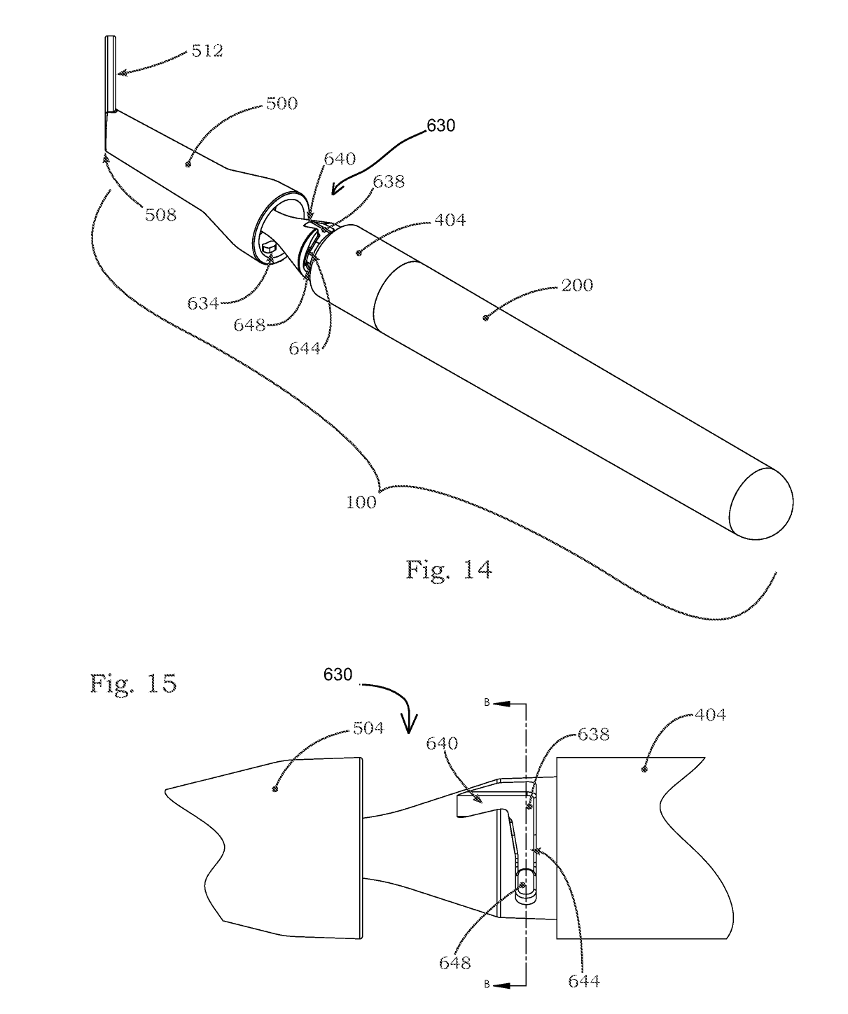

A bayonet style coupling 630 may further use at least one pin-like dog 634 defined by one of the head base 504 and the cap base 404 and at least one cooperating L-shaped groove 638 defined by the other (FIGS. 14-19). With the groove defined by the cap base as shown, the groove may have a stem or first leg 640 aligned along the cap length. A second leg 644 of the groove may extend from the first leg at an end of the leg closest to the body 200. Though not required, the second leg is shown to partially circumscribe the cap. Coupling of the head 500 with the cap 400 is again noted to provide rotational asymmetry in the first order.

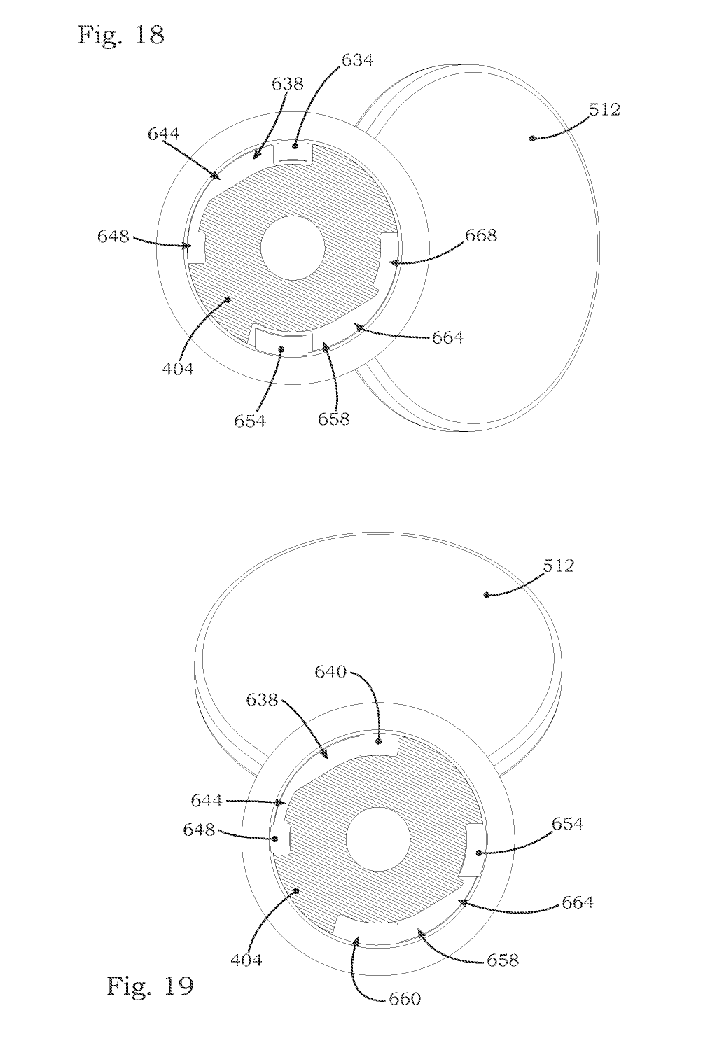

In use, the cap 400 may be sheathed in the head 500 and manipulated by rotation to capture the dog 634 in the stem 640 of the groove 638. So captured, the cap and head may further engage with the dog traveling along the stem 640. Upon the dog coming to the second leg 644 (FIG. 18), the cap and head may again be manipulated by rotation such that the dog 634 travels along the second leg. A detent 648 is preferably provided whereby the dog is releasably captured in the second leg, away from the stem (FIG. 19). One having ordinary skill in the art may devise various viable detents. The detent illustrated in the drawing is only one such detent, comprising a depression or dimple into which the dog sits.

Note is again made as above that a field of view through the mirror 512 preferably coincides with the light “L” emitted from the cap 400. Thus, each of the mirror, the light, the dog 634, and the detent 648 is indexed to provide a relative rotation of the head with the cap in which the field of view through the mirror coincides with the light “L” emitted from the cap when the dog sits in the detent.

A second dog 654 may be defined by one of the head base 504 and the cap base 404 and a second L-shaped groove 658 optionally including a cooperating second detent 668, defined by the other, whereby stability of the coupling may be enhanced. To assure continuing rotational asymmetry in the first order, a thickness of the second dog may be larger as shown or may be smaller, than the first dog 634. Rather depends upon which dog is labeled first. Of course the respective groove is sized accordingly. Another method may include placing the second dog and groove other than diametrically opposite the respective first dog and groove. Accordingly, the second groove may also comprise a stem 660 aligned along the cap length and a second leg 664 extending from the stem at an end of the leg closest to the body 200 such that the second leg 664 may partially circumscribe the cap.

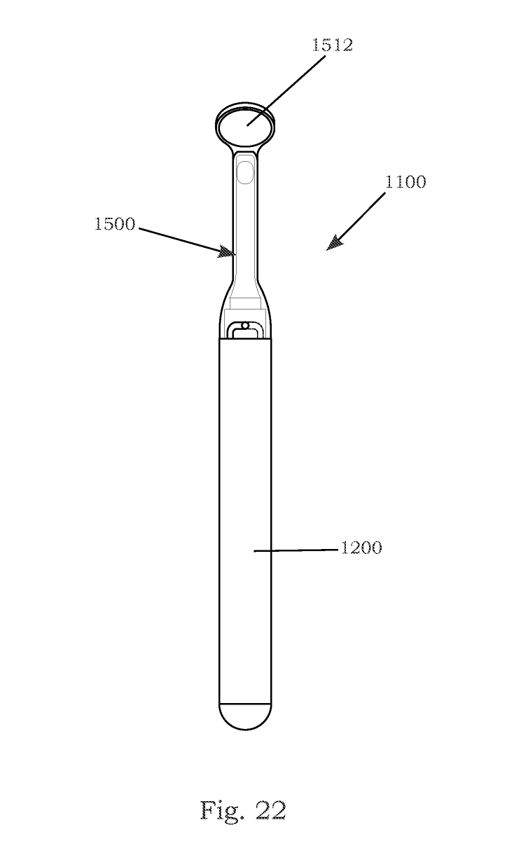

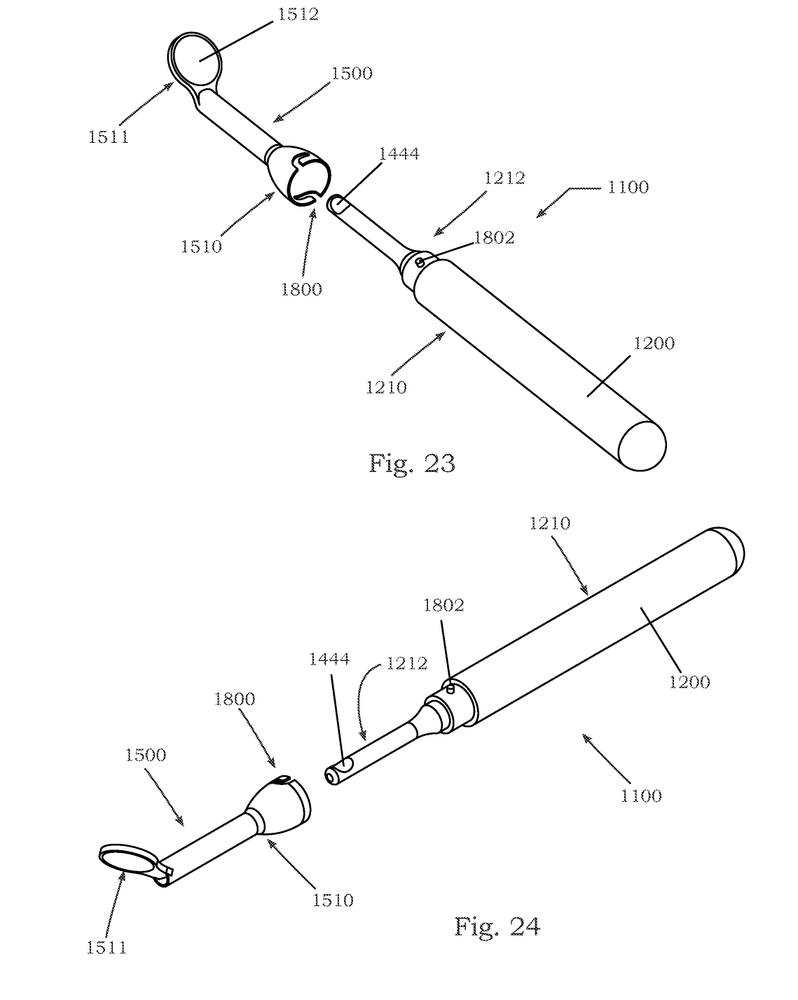

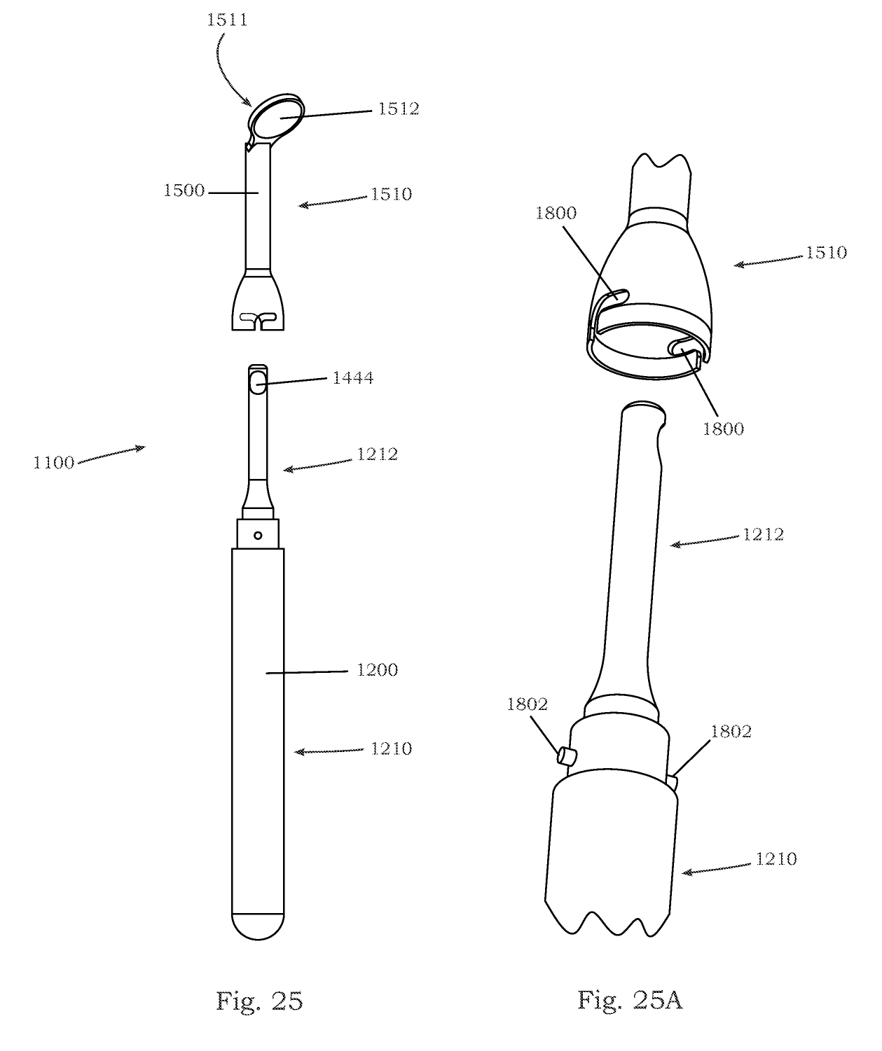

Referring now to the additional embodiment of the illuminated dental mirror 1100 shown in FIGS. 20-25A, an elongated body 1200 similarly has a handle portion 1210 and a tip portion 1212, where a light source is disposed within the elongated body 1200 and is operable to emit light longitudinally along the elongated body 1200 to a deflector 1444, such as a mirror or polished surface or the like, disposed at an interior chamber of the tip portion 1212. A disposable mirror head 1500 is removably attached over the tip portion 1212 using an attachment feature 1800 (FIG. 23) on the mirror head 1500 that removably engages the elongated body in an engaged position that secures and prevents rotation of the mirror head relative to the elongated body. The attachment feature 1800 on the mirror head engages the elongated body in a unique rotational position relative to each other that is configured to align the desired area illuminated by the light source with a user's field of view in the mirror portion of the mirror head. The illustrated attachment feature 1800 has a groove that engages a cooperating dog 1802 protruding laterally from the elongated body, where the groove defines a substantially L-shaped groove, similar to that described above. The details of the dental mirror embodiment 1100 are otherwise similar to that described above with like reference numbers increased by a thousand, so as to generally designate like features of the previously described embodiment, unless describe otherwise above.

One having ordinary skill in the art and those who practice the invention will understand from this disclosure that various modifications and improvements may be made without departing from the spirit of the disclosed inventive concept. One will also understand that various relational terms, including left, right, front, back, top, and bottom, for example, may be used in this detailed description of the invention and in the claims only to convey relative positioning of various elements of the claimed invention without limitation to the invention.

1. A dental mirror comprising:
an elongated body having a handle portion and a tip portion;
a light source disposed within the elongated body and operable to emit light longitudinally along the elongated body to a deflector disposed at an interior chamber of the tip portion, wherein the deflector is configured to redirect light laterally out of the tip portion of the elongated body; and
a disposable mirror head removably attached over the tip portion, wherein the mirror head includes a (i) base portion that comprises a translucent material configured to transmit the light redirected by the deflector to a desired area outside the mirror head and (ii) a mirror portion that extends at an angle from the base portion and that comprises a mirror configured for a user to view the desired area illuminated by the light source.

2. The dental mirror of claim 1, wherein the mirror head includes an attachment feature that removably engages the elongated body in an engaged position that secures and prevents rotation of the mirror head relative to the elongated body.

3. The dental mirror of claim 2, wherein the attachment feature on the mirror head engages the elongated body in a unique rotational position relative to each other that is configured to align the desired area illuminated by the light source with a user's field of view in the mirror portion of the mirror head.

4. The dental mirror of claim 2, wherein the attachment feature comprises a groove that engages a cooperating dog protruding laterally from the elongated body, and wherein the groove defines a substantially L-shaped groove.

5. The dental mirror of claim 1, wherein the base portion of the mirror head includes a recess that corresponds to the tip portion and extends toward the mirror portion, and wherein the base portion in the engaged position covers the tip portion of the elongated body.

6. The dental mirror of claim 1, wherein the base portion of the mirror head has a longitudinal length that is configured to extend into a patient's mouth and position the mirror portion adjacent to a patient's tooth, and wherein the base portion surrounds a circumference of the tip portion along the length of the base portion and comprises an impermeable outer surface that is configured to prevent a patient's saliva from contacting the tip portion of the elongated body.

7. The dental mirror of claim 1, wherein the tip portion includes a diameter that is smaller than a diameter of the handle portion, and wherein an outer diameter of the mirror head substantially aligns with the diameter of the handle portion in the engaged position.

8. The dental mirror of claim 1, wherein a sidewall of the tip portion of the elongated body includes an aperture that extends to a hollow interior chamber of the tip portion for transmitting light redirected laterally from the deflector.

9. The dental mirror of claim 8, wherein the aperture and deflector are arranged to transmit light in a focused area that does not directly contact the mirror portion of the mirror head, and wherein a mirror on the mirror portion is spaced from the handle portion and oriented at an acute angle relative to the handle that is configured to view a patient's tooth illuminated by the focused area of light emitted by the light source.

10. A dental mirror comprising:
an elongated body having a handle portion and a tip portion with a hollow interior chamber;
a light source disposed within the elongated body and operable to emit light toward a distal end of the tip portion;
a lens member coupled with the light source and disposed within the interior chamber of the tip portion, wherein the lens member is configured to transmit light longitudinally along the elongated body from the light source toward the distal end of the tip portion;
a deflector disposed at the interior chamber of the tip portion and configured to redirect light transmitted from the lens member laterally out of the tip portion;
a mirror head removably attached over the tip portion, wherein the mirror head includes a (i) base portion that comprises a translucent material configured to transmit the light redirected by the deflector and (ii) a mirror portion that extends at an angle from the base portion that comprises a mirror configured for a user to view an area illuminated by the light redirected by the deflector through the base portion; and
wherein the mirror head includes an attachment feature that removably engages the elongated body in an engaged position that secures and prevents rotation of the mirror head relative to the elongated body.

11. The dental mirror of claim 10, wherein the attachment feature comprises a groove that engages a cooperating dog protruding laterally from the elongated body.

12. The dental mirror of claim 11, wherein the groove defines a substantially L-shaped groove with a first leg extending longitudinally along the mirror head and a second leg partially circumscribing the base portion of the mirror head.

13. The dental mirror of claim 10, wherein the attachment feature on the mirror head and a corresponding engagement feature on the elongated body couple in a unique rotational position relative to each other that is configured to align the area illuminated by the light source with a field of view of the mirror portion of the mirror head.

14. The dental mirror of claim 10, wherein the tip portion includes a diameter that is smaller than a diameter of the handle portion.

15. The dental mirror of claim 10, wherein the base portion of the mirror head includes a recess that corresponds to at least a portion of the tip portion and extends toward the mirror portion for covering the tip portion of the elongated body.

16. The dental mirror of claim 10 wherein a sidewall of the tip portion of the elongated body includes an aperture that extends to the hollow interior chamber for transmitting light laterally from the elongated body.

17. The dental mirror of claim 16, wherein the aperture and deflector are arranged to transmit light in a focused area that does not directly contact the mirror portion of the mirror head, and wherein a mirror on the mirror portion is spaced from the handle portion and oriented at an acute angle relative to the handle that is configured to view a tooth illuminated by the focused area of light emitted by the light source.

18. A method of using an illuminated dental mirror, said method comprising:
providing an elongated body having a handle portion and a tip portion, wherein a light source is disposed within the elongated body and operable to emit light longitudinally along the elongated body to a deflector disposed at an interior chamber of the tip portion;
removably attaching a disposable mirror head over the tip portion, wherein the mirror head includes a base portion that comprises a translucent material configured to transmit the light redirected laterally by the deflector to a desired area outside the mirror head; and
wherein, when attached in an engaged position, a mirror portion of the mirror head that extends at an angle from the base portion is positioned for a user to view the desired area illuminated by the light source.

19. The method of claim 18, wherein the base portion of the mirror head has a longitudinal length that is configured to extend into a patient's mouth and position the mirror portion adjacent to a patient's tooth for illuminating and viewing the patient's tooth, and wherein the base portion surrounds a circumference of the tip portion along the length of the base portion and comprises an impermeable outer surface that is configured to prevent a patient's saliva from contacting the tip portion of the elongated body.

20. The method of claim 19, wherein the mirror head includes an attachment feature that removably engages the elongated body in the engaged position that secures and prevents rotation of the mirror head relative to the elongated body, and wherein the attachment feature on the mirror head engages the elongated body in a unique rotational position relative to each other that is configured to align the desired area illuminated by the light source with a user's field of view in the mirror portion of the mirror head.
